(19) United States
(12) Patent Application Publication
(10) Pub. No.: US 2022/0313607 A1
(43) Pub. Date: Oct. 6, 2022

(54) LIPOSOMES AND USES THEREOF

(71) Applicant: UNIVERSITÀ DEGLI STUDI DI MILANO - BICOCCA, Milano (MI) (IT)

(72) Inventors: Francesca RE, Milano (MI) (IT); Massimo Ernesto MASSERINI, Milano (MI) (IT); Marco Antonio Ercole SARDINA, Milano (MI) (IT)

(73) Assignee: UNIVERSITÀ DEGLI STUDI DI MILANO - BICOCCA, Milano (MI) (IT)

(21) Appl. No.: 17/630,681

(22) PCT Filed: Jul. 31, 2020
(86) PCT No.: PCT/EP2020/071683

§ 371 Date: Jan. 27, 2022

(30) Foreign Application Priority Data
Jul. 31, 2019 (IT) 102019000013569

(51) Int. Cl. A61K 9/127; A61K 47/28; A61K 47/24; A61K 38/17; A61P 25/28

(52) U.S. Cl. CPC A61K 9/1272; A61K 47/28; A61K 47/24; A61K 38/17; A61P 25/28

(57) ABSTRACT

The present invention relates to a liposome containing a lipid or lipid mixture, phosphatidic acid and/or cardiolipin and apolipoprotein E for use in the treatment and/or prevention of amyloidosis, wherein the amyloidosis is not Alzheimer's disease and pharmaceutical compositions containing the same.

### TECHNICAL FIELD

The present invention relates to a liposome comprising a lipid or lipid mixture, phosphatidic acid and/or cardiolipin and apolipoprotein E for use in the treatment and/or prevention of amyloidosis, wherein said amyloidosis is not Alzheimer's disease and relative pharmaceutical compositions.

### BACKGROUND ART

Amyloidosis is a group of diseases in which abnormal proteins, known as amyloid fibrils, build up in tissues [Chiti F, Dobson CM Annual Reviews of Biochemistry, 2017, 86, 27]. Symptoms depend on the type of disease and are often variable. There are about 30 different types of amyloidosis, each due to a specific protein misfolding. Some are genetic while others are acquired. They are grouped into localized and systemic forms: localised amyloidoses affect only one body organ or tissue type, e.g. Cerebral Amyloid Angiopathy; systemic amyloidoses affect more than one body organ or system, e.g. light chain (AL, Amyloidosis due to antibodies light chain), inflammation (AA, Amyloidosis due to protein A), dialysis (Aβ₂M, Amyloidosis due to β₂Microglobulin), and hereditary and old age (ATTR, Amyloidosis due to Transthyretin). Although amyloidogenic proteins have heterogeneous structures and functions and the causes of amyloid-associated diseases vary, all these proteins can generate amyloid fibrils. Without treatment, life expectancy is between six months and four years —about 1 of 1.000 in the developed world die of Amyloidosis.

Few treatments are available against amyloidosis, e.g high-dose chemotherapy, anti-inflammatory drugs (e.g. steroids, anti-TNF) and immunosuppressants, but none of them is completely resolutive the disease. With hereditary Amyloidosis, due to mutations of the genes encoding for amyloidogenic proteins, transplants of liver, kidneys or heart are often the last option.

β-amyloid peptide (Aβ40) is a 40-residue proteolytic product of the much larger amyloid precursor protein (APP) encoded by a gene on chromosome 21. The deposition of Aβ40 in the tunica media and adventitia of the arterioles and/or capillaries in the cerebral cortex and leptomeninges is one of the main pathological features of the Cerebral amyloid angiopathy (CAA). CAA is the second cause (after hypertension) causing cerebral hemorrhage in the elderly, accounting for 15-40% of non-traumatic cerebral haemorrhage in the elderly with a mortality of 30-50%. Occasionally, CAA can be presented as cerebral ischemic attack, cognitive dysfunction, and cerebral vasculitis. In addition, CAA is commonly found in Alzheimer's disease (AD) and nearly 80% of AD patients are accompanied by CAA.

Transthyretin (TTR) is a homotetrameric, β-sheet-rich protein of 56 kDa produced in the liver or in cerebral spinal fluid (CSF). TTR is associated with two amyloidosis: senile systemic amyloidosis (SSA) and familial amyloid polyneuropathy (FAP). SSA is caused by the massive deposition of aggregates composed of wild-type TTR (WT-TTR) mainly in the heart, while FAP is caused by the deposition of aggregates at the peripheral nerves and tissues that are composed of more than 100 autosomal TTR variants. β-₂ microglobulin (β2m) is the light chain of class I major histocompatibility complex; it is composed of 99 residues, organized in a classic β-sandwich structure, consisting of two β-sheets locked together by a disulphide bond. β2m in vivo aggregation is responsible for dialysis-related amyloidosis, a pathological state that affects patients undergoing extended hemodialysis periods. Many β2m mutational studies have been performed to elucidate which molecular properties affect β2m aggregation. As for example, the amyloidogenic variant of β2-microglobulin, D76N, is associated with a familial form of the disease and is characterized by progressive bowel disfunction and extensive amyloid deposits in the spleen, liver, heart, salivary glands and nerves [Valleix S. et al. N Engl J Med. 2012 Jun 14; 366(24):2276-83]. ΔN6 is a ubiquitous constituent of β2-m amyloid deposits in patients affected by dialysis-related amyloidosis and, due to its capacity to act as a seed in the fibrillogenesis of full length β2-m, it could have a crucial role in dictating the clinical history of the disease [Esposito G. et al. Protein Sci. 2000 May; 9(5):831-45].

Protein serum amyloid A (SAA) accumulates in organs like the spleen, liver, and kidney as amyloid deposits, originating a condition called reactive amyloidosis or Amyloid A (AA) amyloidosis. Reactive amyloidosis generally accompanies other conditions that induce chronic inflammation such as rheumatoid arthritis and atherosclerosis. SAA deposits have also been observed in type 2 diabetes [Anderberg RJ et al. Laboratory Investigation 2015, 95, 250]l

Therefore, there is still the need for new therapies for the treatment of amyloidosis.

### SUMMARY OF THE INVENTION

In the present invention it is surprisingly shown that liposomes comprising phosphatidic acid and/or cardiolipin, apoliprotein E and lipid or lipid mixture are able to interact with different amyloidogenic proteins than Aβ-42, thus affecting their aggregation, either slowing down or preventing their aggregation into fibrils or inducing the disaggregation of their fibrillary aggregates. Surprisingly, the amyloidogenic proteins are unrelated in reference to their biological function or to their amino acid sequences.

The results herein reported show that the present liposomes induce disaggregation of fibrils, accompanied with a reduction of fibrils order and geometry. The dissolution, or reduction of amyloid aggregates with formation of more soluble species induced by the present liposomes indicates that they are suitable for the pharmacological treatment of amyloidosis.

Moreover, the results herein reported also show that the liposomes of the present invention prevent or slow down the transition from non-aggregated to fibrillar state of amyloidogenic proteins, indicating that liposomes object of the present invention are suitable for pharmacological treatment of early stage amyloidosis.

In particular, the liposomes of the present invention:

i) inhibit TTR aggregation by 60% and completely induce its disaggregation;
ii) completely inhibit β2 microglobulin aggregation and partially induce its microglobulin disaggregation;
iii) completely inhibit Aβ40 aggregation and completely induce its disaggregation;
iv) completely inhibit SAA1-76 aggregation and induce its disaggregation;
v) bind the aggregates of Aβ40, β2MΔN6,β2MD76N and TTR and
vi) reduce vascular deposition of Aβin animal model of CAA.

Then, the present liposomes are particularly suitable for the treatment and/or prevention of amyloidosis at all stages of the disease.

Therefore, the present invention provides a liposome comprising:

a lipid or lipid mixture;
phosphatidic acid and/or cardiolipin and
apolipoprotein E or a fragment thereof for use in the treatment and/or prevention of amyloidosis, wherein said amyloidosis is not Alzheimer's disease and is selected from the group consisting of: senile systemic amyloidosis, familial amyloid polyneuropathy, dialysis-related amyloidosis, reactive amyloidosis, cerebral amyloid angiopathy, prion diseases such as Creutzfeldt—Jakob disease (humans), BSE or “mad cow disease” (cattle), and scrapie, Finnish type amyloidosis, leptomeningeal amyloidosis, Familial visceral amyloidosis, Primary cutaneous amyloidosis, Prolactinoma, Familial corneal amyloidosis, Senile amyloid of atria of heart, Medullary carcinoma of the thyroid LECT2 amyloidosis, type 2 diabetes mellitus, end stage renal failure in patients with type 1 and 2 diabetes mellitus and diabetic kidney disease, preferably when the reactive amyloidosis is accompanied by rheumatoid arthritis and/or atherosclerosis and wherein said lipid or lipid mixture is selected from the group consisting of: sphingomyelin, phosphatidylcholine, phosphatidylethanolamine and cholesterol.

Preferably the lipid is a mixture of sphingomyelin and cholesterol, still preferably in a 1:1 molar ratio.

Preferably the liposome does not comprise monosialoteterahexosyl ganglioside (GM1). The term “phosphatidic acid” used herein means a molecule having a glycerol backbone esterified with two fatty acids and one phosphate group, for example dimiristoylphosphatidic acid. Cardiolipin is a well-known compound, also known as I,3-bis(sn-3′-phosphatidyl)-sn-glycerol. In the liposome, the phosphatidic acid (or cardiolipin) is preferably present in 1-20%, more preferably 1-10%, most preferably 5% molar amount (with respect to the total moles of all substances making up the liposome). If phosphatidic acid or cardiolipin are both present, the above ranges are referred to the sum of moles of both substances; when both present, phosphatidic acid and cardiolipin can be used in any mutual ratio.

Apolipoprotein E (ApoE) is a 34 kDa glycoprotein containing 299 aminoacids, produced in high levels in liver and brain. ApoE can be used as obtained from its natural sources or be synthetized on purpose. The term “ApoE derivatives” includes the known ApoE isoforms, coded E2, E3. The term “ApoE derivative” also includes fragments of ApoE; preferably those belonging to the aminoacid sequence 100-200, more preferably 120-170; a preferred fragment is the one consisting in the aminoacids 141-150 of ApoE (Sequence: (LRKLRKRLLR) —N H 2, SEQ ID No. 10) or dimer thereof (Sequence: (LRKLRKRLLR)—(LRKLRKRLLR)—NH SEQ ID No. 11). Preferably, the ApoE or fragment thereof includes a terminal, cystein-ending, small peptide sequence (up to five aminoacids), such as the tripeptide CWG-, assisting in the chemical linkage to the lipid: preferred examples of the resulting fragments are: CWG-(LRKLRKRLLR)-NH2 SEQ ID No. 1 , herein “mApoE” , or C WG-(LRKLRKRLLR)-(LRKLRKRLLR)-NH2 SEQ ID No. 12 , herein “dApoE”.

Said ApoE or derivative thereof may be present in the liposomes as a physical mixture with the other lipids constituents of the liposome membrane, or be deposited on such membrane, or be chemically linked to these lipids, or be contained within the liposome, or be added to existing liposomes, etc. Preferably, it is chemically linked. In this case, the ApoE as above described may be linked or connected via a linker molecule; a preferred linker is the compound 1,2 stearoyl-sn-glycero-3- phosphoethanolamine-N-[maleim ide(poly(ethylene glycol)-2000)] (herein abbreviated “mal-PEG-PE”). In the final liposome, the ApoE is preferably present in a molar amount of 1-5% (with respect to the total moles of all substances making up the liposome). The final liposome further comprises standard liposome lipids. These make up the bulk of the liposome, preferably accounting for a 90-98% molar amount (with respect to the total moles of all substances making up the liposome). Preferred standard liposome lipids are sphingomyelin, phosphatidylcholine, phosphatidylethanolamine (PEGylated or not) and cholesterol.

In a preferred embodiment the apolipoprotein E comprises the two known isoforms E2, E3 of ApolipoproteinE and fragments thereof, preferably the fragments selected from the aminoacid sequence 100-200 of ApoE; more preferably within the aminoacid sequence 120-170 of ApoE; most preferably the apolipoprotein E is the sequence 141-150 or a dimer thereof.

Preferably said apolipoprotein E includes, at its C-terminal, a cystein-ending tripeptide preferably being the tripeptide CWG-, preferably the apolipoprotein E has the sequence CWGLRKLRKRLLR or is a dimer thereof.

In a preferred embodiment the liposome further comprises at least one PEG (polyethyleneglycol) molecule, PEO (poly-ethylene-oxide) molecule, POE (poly-oxy-ethylene) molecule, PDO (Polydioxanone) molecule or a mixture thereof, preferably the average molecular mass of the PEG molecule is above 1 kDa but less than 11 kDa.

Preferably the PEG molecule is selected from the group consisting of: methylpolyethyleneglycol-1,2-distearoyl-phosphatidyl ethanolamine conjugate (MPEG-2000-DSPE); monomethoxypolyethylene glycol (MPEG-OH), monomethoxypolyethylene glycol-succinate (MPEG-S), monomethoxypolyethylene glycol-succinim idyl succinate (MPEG-S-NHS), monomethoxypolyethylene glycol-amine (MPEG-NH2), monomethoxypolyethylene glycol-tresylate (MPEG-TRES), and monomethoxypolyethylene glycol-imidazolyl-carbonyl (MPEG-IM); or mixtures thereof.

Preferably the phosphatidic acid is present in 1-20% molar percentage, preferably 1-10%, most preferably 5% molar percentage.

Still preferably the apolipoprotein E is present in 1-5% molar percentage, preferably 1-3%, most preferably 1.25% molar percentage.

In a preferred embodiment said liposome consists of:

46,25 mol % cholesterol
46,25 mol % sphingomyelin
1.25-1,5% mol mal-PEG-PE linked to mApoE
1.25-1,0% mol mal-PEG-PE free
5 mol % phosphatidic acid wherein the sum of the % of mal-PEG-PE free and % mal-PEG-PE linked to mApoE is 2,5%.

Preferably the liposome of the present invention has an average size <200 nm. Yet preferably the liposome has a PDI <0.2, preferably <0.1.

The Liposome polydispersity (PDI) may be measured by any known method in the art such as Dynamic Laser light scattering (DLS) and was obtained from the intensity autocorrelation function of the light scattered at a fixed angle of 90 degrees. The correlation function was analyzed by means of a two-cumulant expansion. Each measurement was performed under an electrical field of 29.7 V/cm ([Gobbi M, et al. Biomaterials 2010;31:6519-29)].

In a preferred embodiment the liposome decreases amyloid protein aggregation and/or increases amyloid protein disaggregation in respect to an amyloid protein aggregation without liposome.

Preferably the amyloid protein is selected from the group consisting of: Transthyretin , β₂microglobulin, amyloid light chain, Serum amyloid A protein, Islet amyloid peptide, Gelsolin, Cystatin C, ApoA1, Fibrinogen alfa chain, LYZ (Lysozyme, also known as muramidase or N-acetylmuramide glycanhydrolase), OSMR (Oncostatin-M specific receptor subunit beta also known as the Oncostatin M receptor), Integral membrane protein 2B (ITM2B or BRI2), prolactin, LECT2 protein, keratoepithelin (Transforming growth factor, beta-induced, 68 kDa, also known as TGFBI (initially called BIGH3, BIG-H 3), calcitonin, atrial natriuretic factor and prion protein.

In a preferred embodiment the amyloidosis is selected from the group consisting of: senile systemic amyloidosis, familial amyloid polyneuropathy, dialysis-related amyloidosis, reactive amyloidosis, cerebral amyloid angiopathy, prion diseases such as Creutzfeldt—Jakob disease (humans), BSE or “mad cow disease” (cattle), and scrapie, Finnish type amyloidosis, leptomeningeal amyloidosis, Familial visceral amyloidosis, Primary cutaneous amyloidosis, Prolactinoma, Familial corneal amyloidosis, Senile amyloid of atria of heart, Medullary carcinoma of the thyroid LECT2 amyloidosis, type 2 diabetes mellitus, end stage renal failure in patients with type 1 and 2 diabetes mellitus and diabetic kidney disease, preferably the reactive amyloidosis is accompanied by rheumatoid arthritis and/or atherosclerosis.

The present invention also provides a formulation comprising the liposome according to any one of previous claim for use in the treatment and/or prevention of amyloidosis, wherein said amyloidosis is not Alzheimer's disease and is selected from the group consisting of: senile systemic amyloidosis, familial amyloid polyneuropathy, dialysis-related amyloidosis, reactive amyloidosis, cerebral amyloid angiopathy, prion diseases such as Creutzfeldt-Jakob disease (humans), BSE or “mad cow disease” (cattle), and scrapie, Finnish type amyloidosis, leptomeningeal amyloidosis, Familial visceral amyloidosis, Primary cutaneous amyloidosis, Prolactinoma, Familial corneal amyloidosis, Senile amyloid of atria of heart, Medullary carcinoma of the thyroid LECT2 amyloidosis, type 2 diabetes mellitus, end stage renal failure in patients with type 1 and 2 diabetes mellitus and diabetic kidney disease, preferably the reactive amyloidosis is accompanied by rheumatoid arthritis and/or atherosclerosis.

Preferably the liposome of the present invention may be used in combination with existing treatment or therapeutic intervention of amyloidosis known to the skilled person in the art, such as a chemotherapy agent (for instance high dose melphalan), stem cell transplantation, steroids, eprodisate, liver transplant, patisiran and as described for instance in Wechalekar AD Lancet. 2016 Jun 25;387(10038):2641-2654. doi: 10.1016/S0140-6736(15)01274-X; Ankarcrona M J Intern Med. 2016 Aug;280(2):177-202. doi: 10.1111/joim.12506. Nuvolone M Expert Opin Ther Targets. 2017 Dec;21(12):1095-1110, incorporated by reference.

In the present invention amyloidosis include all amyloidosis except Alzheimer's disease.

Amyloidosis is a group of diseases in which abnormal proteins, known as amyloid fibrils, build up in tissue. Symptoms depend on the type and are often variable. They may include diarrhoea, weight loss, feeling tired, enlargement of the tongue, bleeding, numbness, feeling faint with standing, swelling of the legs, or enlargement of the spleen.

The presentation of amyloidosis is broad and depends on the site of amyloid accumulation. The kidney and heart are the most common organs involved.

Amyloid deposition in the kidneys can cause nephrotic syndrome, which results from a reduction in the kidney's ability to filter and hold on to proteins. The nephrotic syndrome occurs with or without elevations in creatinine and blood urea concentration, two biochemical markers of kidney injury. In AA amyloidosis, the kidneys are involved in 91-96% of patients, symptoms ranging from protein in the urine to nephrotic syndrome and rarely chronic kidney disease.

Amyloid deposition in the heart can cause both diastolic and systolic heart failure. EKG changes may be present, showing low voltage and conduction abnormalities like atrioventricular block or sinus node dysfunction. On echocardiography, the heart shows a restrictive filling pattern, with normal to mildly reduced systolic function. AA amyloidosis usually spares the heart.

People with amyloidosis can develop sensory and autonomic neuropathies. Sensory neuropathy develops in a symmetrical pattern and progresses in a distal to proximal manner. Autonomic neuropathy can present as orthostatic hypotension but may manifest more gradually with nonspecific gastrointestinal symptoms like constipation, nausea, or early satiety.

Accumulation of amyloid proteins in the liver can lead to elevations in serum aminotransferases and alkaline phosphatase, two biomarkers of liver injury, which is seen in about one third of people. Liver enlargement is common. In contrast, spleen enlargement is rare, occurring in 5% of people. Splenic dysfunction, leading to the presence of Howell-Jolly bodies on blood smear, occurs in 24% of people with amyloidosis. Malabsorption is seen in 8.5% of AL amyloidosis and 2.4% of AA amyloidosis. One suggested mechanism for the observed malabsorption is that amyloid deposits in the tips of intestinal villi (fingerlike projections that increase the intestinal area available for absorption of food), begin to erode the functionality of the villi, presenting a sprue-like picture.

A rare development is amyloid purpura, a susceptibility to bleeding with bruising around the eyes, termed “raccoon-eyes”, caused by amyloid deposition in the blood vessels and a reduced activity of thrombin and factor X, two clotting proteins that lose their function after binding with amyloid.

Amyloid deposits in tissue can cause enlargement of structures. Twenty percent of people with AL amyloidosis have an enlarged tongue, that can lead to obstructive sleep apnea, difficulty swallowing, and altered taste. Tongue enlargement does not occur in ATTR or AA amyloidosis. Enlarged shoulders, “shoulder pad sign”, results from amyloid deposition in the synovial space. Deposition of amyloid in the throat can cause hoarseness. Aβ2MG amyloidosis (Hemodialysis associated amyloidosis) tends to deposit in synovial tissue, causing chronic inflammation of the synovial tissue, which can lead to repeated carpal tunnel syndrome.

Both the thyroid and adrenal glands can be infiltrated. It is estimated that 10-20% of individuals with amyloidosis have hypothyroidism. Adrenal infiltration may be harder to appreciate given that its symptoms of orthostatic hypotension and low blood sodium concentration may be attributed to autonomic neuropathy and heart failure.

Amyloid deposits occur in the pancreas of patients with diabetes mellitus. The major component of pancreatic amyloid is a 37-amino acid residue peptide known as islet amyloid polypeptide or amylin. This is stored with insulin in secretory granules in B cells and is co secreted with insulin (Rang and Dale's Pharmacology, 2015.)

Uncommonly, a collection of amyloid can grow large enough to be classed as an amyloidoma, a macroscopic lump of amyloid that can cause mass effect.

There are about 30 different types of amyloidosis, each due to a specific protein misfolding. Some are genetic while others are acquired. They are grouped into localized and systemic forms. The four most common types of systemic disease are light chain (AL), inflammation (AA), dialysis (Aβ2M), and hereditary and old age (ATTR).

Brief description of the more common types of amyloid:

| | Amyloid | | |
|---|---|---|---|
| Abbr. | type/Gene | Description | OMIM |
| AL | amyloid light | AL amyloidosis/multiple myeloma. | 254500 |
| | chain | Contains immunoglobulin light-chains (λ, κ) derived | |
| | | from plasma cells. | |
| AA | SAA | Serum amyloid A protein (SAA) is an acute-phase | |
| | | reactant that is produced in times of inflammation. | |
| ALECT2 | LECT2 | In LECT2 amyloidosis, the LECT2 protein deposits in | |
| | | the kidneys and various other tissues but only kidneys | |
| | | show signs or symptoms; these are typical those of | |
| | | kidney failure. | |
| ATTR | transthyretin | Transthyretin is a protein that is mainly formed in the | 105210 |
| | | liver that transports thyroxine and retinol binding | |
| | | protein. A mutant form of a normal serum protein that | |
| | | is deposited in the genetically determined familial | |
| | | amyloid polyneuropathies. TTR is also deposited in the | |
| | | heart in wild-type transthyretin amyloidosis, also known | |
| | | as senile systemic amyloidosis. Also found | |
| | | in leptomeningeal amyloidosis. | |
| Aβ₂M | β₂microglobulin | Not to be confused with Aβ, β₂m is a normal serum | |
| | | protein, part of major histocompatibility complex (MHC) | |
| | | Class 1 molecules. Haemodialysis-associated | |
| | | amyloidosis | |
| AIAPP | amylin | Found in the pancreas of people with type 2 diabetes. | |
| APrP | prion protein | In prion diseases, misfolded prion proteins deposit in | 123400 |
| | | tissues and resemble amyloid proteins. Some | |
| | | examples are Creutzfeldt-Jakob | |
| | | disease (humans), BSE or “mad cow disease” (cattle), | |
| | | and scrapie (sheep and goats). A recently described | |
| | | familial prion disease presents with peripheral | |
| | | amyloidosis causing autonomic neuropathy and | |
| | | diarrhea. | |
| AGel | GSN | Finnish type amyloidosis | 105120 |
| ACys | CST3 | Cerebral amyloid angiopathy, Icelandic-type | 105150 |
| AApoA1 | APOA1 | Familial visceral amyloidosis | 105200 |
| AFib | FGA | Familial visceral amyloidosis | 105200 |
| ALys | LYZ | Familial visceral amyloidosis | 105200 |
| ? | OSMR | Primary cutaneous amyloidosis | 105250 |
| ABri | ITM2B | Cerebral amyloid angiopathy, British-type | 176500 |
| ADan | | Danish-type | 117300 |
| APro | prolactin | Prolactinoma | |
| AKer | keratoepithelin | Familial corneal amyloidosis | |
| AANF | atrial natriuretic | Senile amyloid of atria of heart | |
| | factor | | |
| ACal | calcitonin | Medullary carcinoma of the thyroid | |

As of 2010, 27 human and 9 animal fibril proteins were classified, along with 8 inclusion bodies.

An older clinical method of classification refers to amyloidoses as systemic or localised Systemic amyloidoses affect more than one body organ or system. Examples are AL, AA and Aβ2m.

Localised amyloidoses affect only one body organ or tissue type. Examples are Aβ, IAPP, Atrial natriuretic factor (in isolated atrial amyloidosis), and Calcitonin (in medullary carcinoma of the thyroid)

Another classification is primary or secondary. Primary amyloidoses arise from a disease with disordered immune cell function, such as multiple myeloma or other immunocyte dyscrasias. Secondary (reactive) amyloidoses occur as a complication of some other chronic inflammatory or tissue-destroying disease. Examples are reactive systemic amyloidosis and secondary cutaneous amyloidosis.

Additionally, based on the tissues in which it is deposited, it is divided into mesenchymal (organs derived from mesoderm) or parenchymal (organs derived from ectoderm or endoderm).

The above described liposomes are per se fully effective on the amyloid deposits in-vivo, as herein demonstrated; as an option, they may additionally include further active agents, also useful for the intended treatment; in such case the present liposomes perform the double function of drug and drug carrier.

A further object of the invention are pharmaceutical compositions comprising the above described liposomes, together with one or more pharmaceutically acceptable excipients and, optionally, further active agents. The type and amounts of excipients are chosen in function of the chosen pharmaceutical form; suitable pharmaceutical forms are liquid systems like solutions, infusions, suspensions; semisolid systems like colloids, gels, pastes or cremes; solid systems like powders, granulates, tablets, capsules, pellets, microgranulates, minitablets, microcapsules, micropellets, suppositories; etc. Each of the above systems can be suitably be formulated for normal, delayed or accelerated release, using techniques well-known in the art.

The liposome are especially active in inhibiting (i.e. preventing, reducing or eliminating; eliminating being preferred) in-vivo the amyloid deposits or aggregates. This activity is useful in patients suffering from or being at risk of developing the above diseases. The invention thus includes therapeutic methods and medical uses aimed at inhibiting in-vivo the amyloid protein deposit or aggregation in patients suffering from or being at risk of developing the above diseases, characterized by the administration of the above described liposomes or pharmaceutical compositions.

The invention further includes in vitro methods to provide an enhanced reduction of amyloid protein deposits, characterized by including ApoE or a derivative thereof in a liposome containing phosphatidic acid and/or cardiolipin, taking advantage from the unexpected finding that these components cooperate synergically in inhibiting the aggregation of amyloid peptides into fibrils and plaques and stimulate disaggregation of preexisting aggregates. The above treatments can be performed by delivering the above liposomes/pharmaceutical compositions to a patient in need thereof, in suitable dose unit, via any suitable administration route. Suitable dose units are comprised in the 1-15 mmoles total lipids for an average 70 kg patient. All administration routes (enteral, parenteral) enabling a systemic distribution of the medicament are contemplated.

Example of possible administration routes are: oral, intravenous, intramuscular, inhalatory, intratracheal, intraperitoneal, buccal, sublingual, nasal, subcutaneous, transdermal, transmucosal. In the present invention, the administration routes directly into the central nervous system (i.e. into those areas placed beyond the blood brain barrier) are definitely not necessary for the present liposomes, since they are advantageously active in the CNS via simple systemic administration. From the therapeutic perspective it is primarily important that the treatment with the present liposomes obtains a strong in-vivo reduction of the amyloid plaque via systemic administration, by means of a low-toxicity active agent, exempt from side effects, at moderate dosages. A long-wanted in-vivo effective treatment is thus provided, easy-to-perform, involving a non-expensive process of preparation, useful for the treatment and prevention of diseases depending from the formation of amyloid protein deposits.

The present invention will be illustrated by means of non-limiting examples in reference to the following figures.

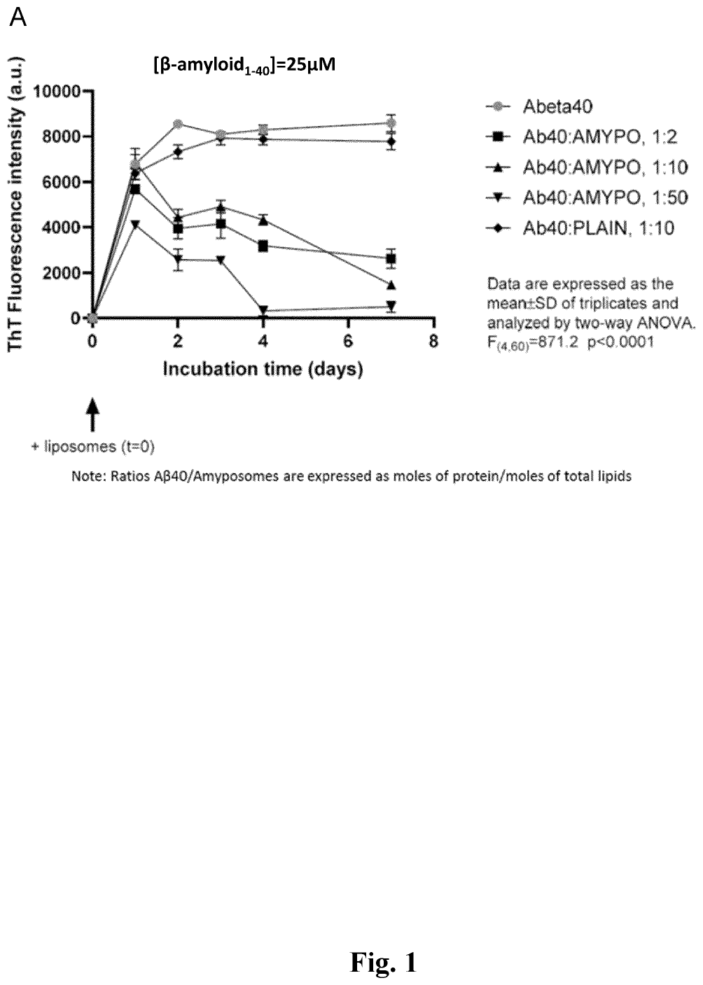

FIG. 1: Inhibition of Aβ-40 in-vitro aggregation of at different protein:Amyposomes ratios, investigated by ThioflavinT fluorescence spectroscopy. (A) time course of ThT fluorescence. a.u. =Fluorescence Intensity arbitrary units.

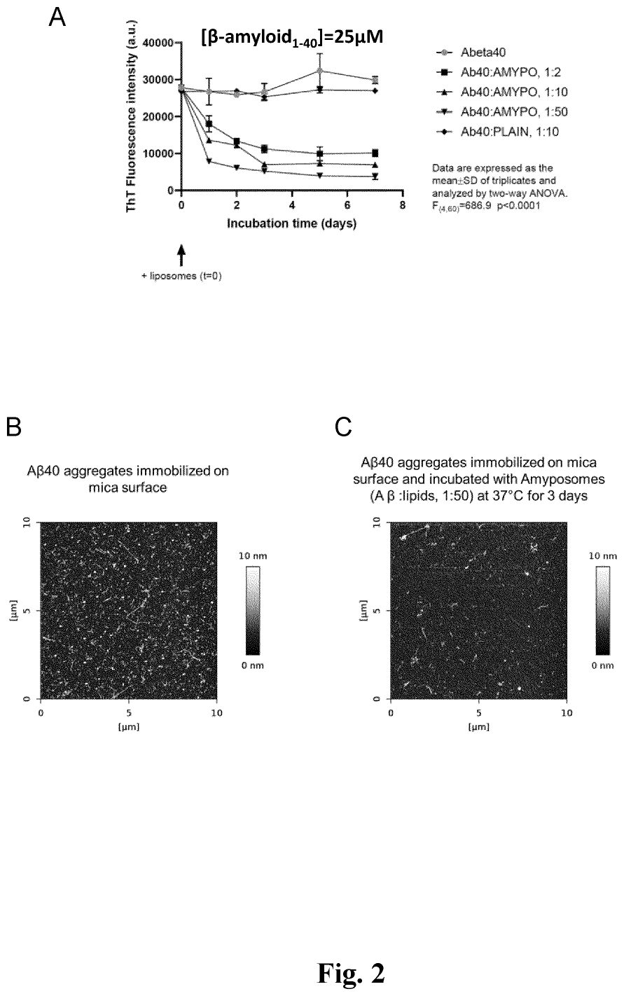

FIG. 2: Induction of Aβ40 fibrils disaggregation in-vitro at different protein:Amyposomes ratios, investigated by ThioflavinT fluorescence spectroscopy. (A) time course of ThT fluorescence. (B) 10 μI of 25 μM Amyloid β-40 fibrils seeded on AFM mica surface and imaged by AFM. (C) 10 μI of 25 μM Amyloid β- 40 fibrils seeded on AFM mica surface, incubated with Amypsomes at 1:50 (M:M) ratio for 3 days and imaged with AFM. a.u. =Fluorescence Intensity arbitrary units.

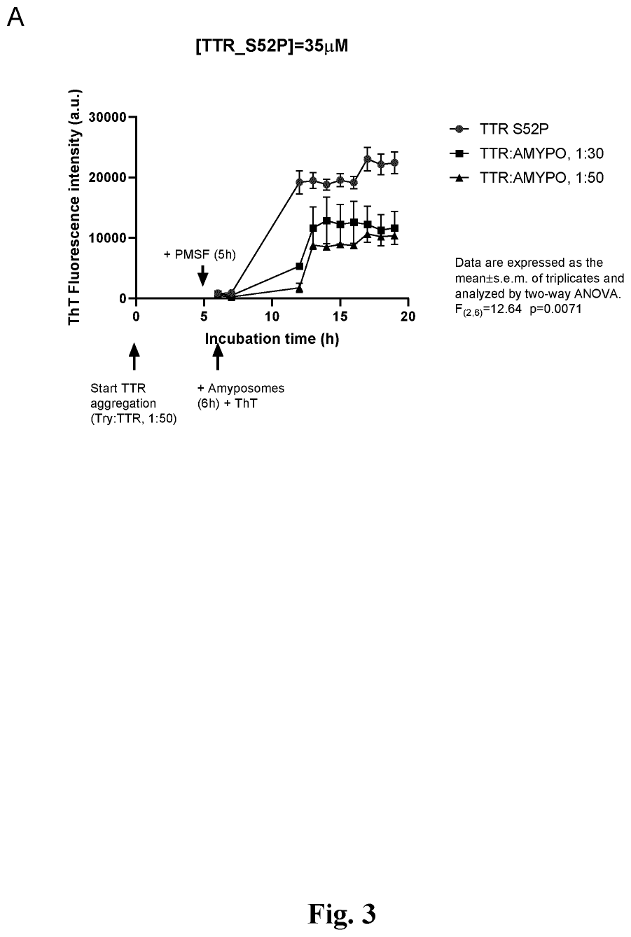

FIG. 3: Inhibition of TTR in-vitro aggregation at different protein:Amyposomes ratios, investigated by ThioflavinT fluorescence spectroscopy. (A) time course of ThT fluorescence. a.u. =Fluorescence Intensity arbitrary units.

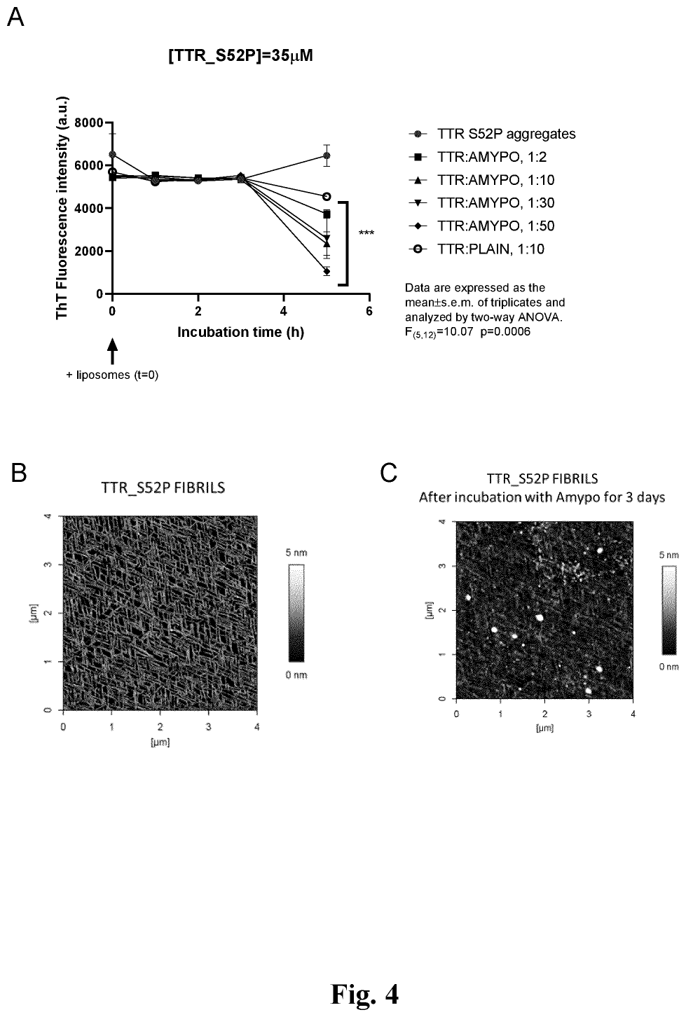

FIG. 4: Induction of TTR fibrils disaggregation in vitro at different protein:Amyposomes ratios, investigated by ThioflavinT fluorescence spectroscopy. (A) time course of ThT fluorescence. (B) 10 μL of 40 μM TTR fibrils seeded on mica surface imaged by AFM. (C) 10 μL of 40 μM TTR fibrils seeded on AFM mica surface, incubated with Amyposomes at 1:50 (M:M) ratio for 3 days and imaged with AFM. a.u. =Fluorescence Intensity arbitrary units.

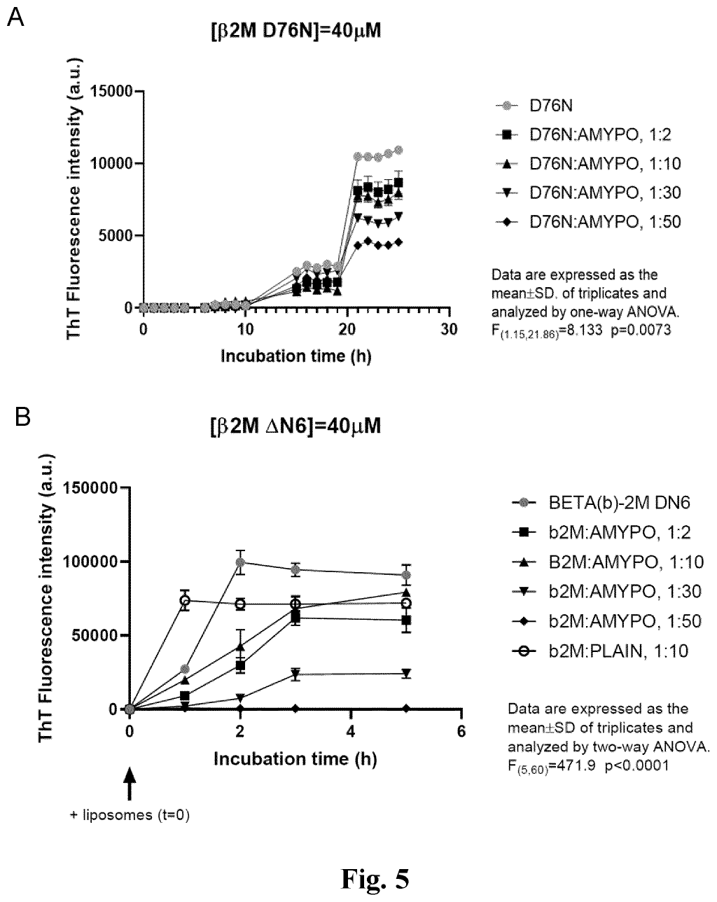

FIG. 5: Inhibition of in-vitro β2 Microglobulin aggregation at different protein:Amyposomes ratios, investigated by ThioflavinT fluorescence spectroscopy. (A) time course of ThT fluorescence for D76N variant. (B) Time course of ThT fluorescence for ΔN6 variant. a.u. β2 Fluorescence Intensity arbitrary units.

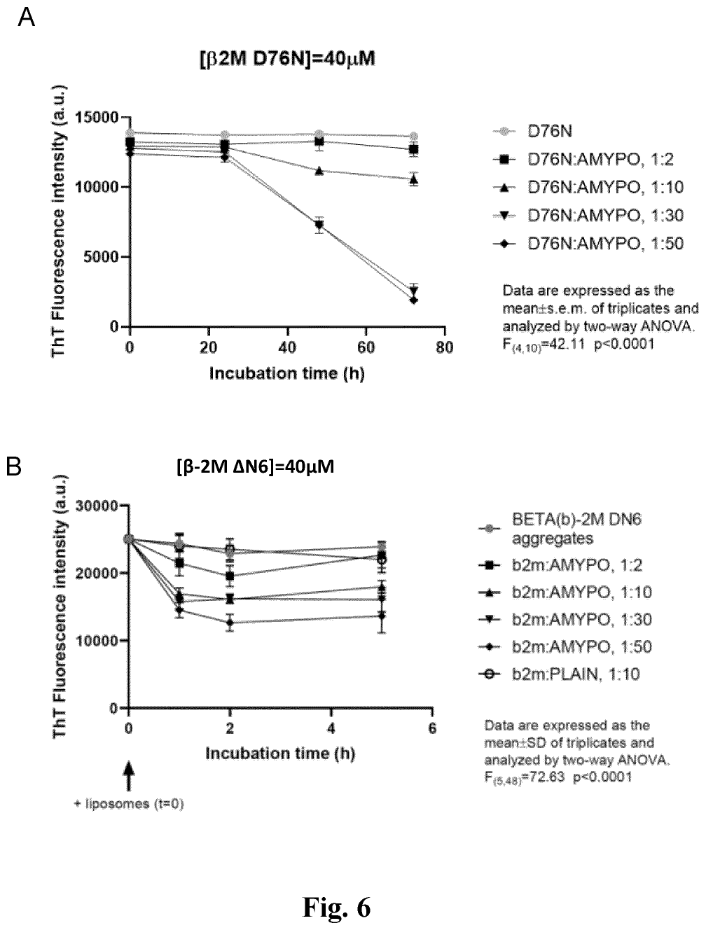

FIG. 6: Induction of β2 Microglobulin fibrils disaggregation in-vitro at different protein:Amyposomes ratios, investigated by ThioflavinT fluorescence spectroscopy. (A) time course of ThT fluorescence for D76N variant. (B) Time course of ThT fluorescence for AN6 variant. a.u. =Fluorescence Intensity arbitrary units.

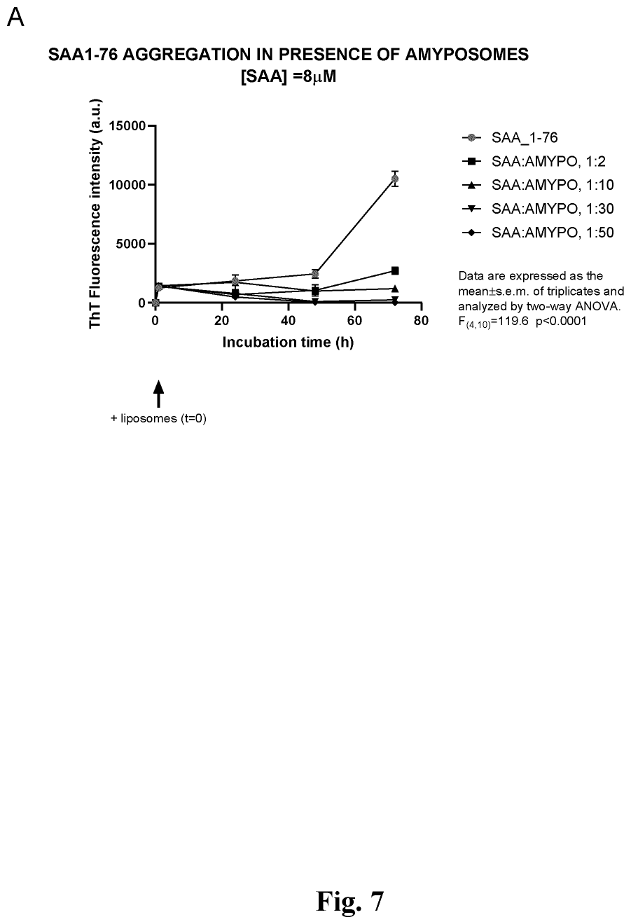

FIG. 7: Induction of SAA(1-76) fibrils disaggregation in-vitro at different protein:Amyposomes ratios, investigated by ThioflavinT fluorescence spectroscopy. (A) time course of ThT fluorescence. a.u. =Fluorescence Intensity arbitrary units.

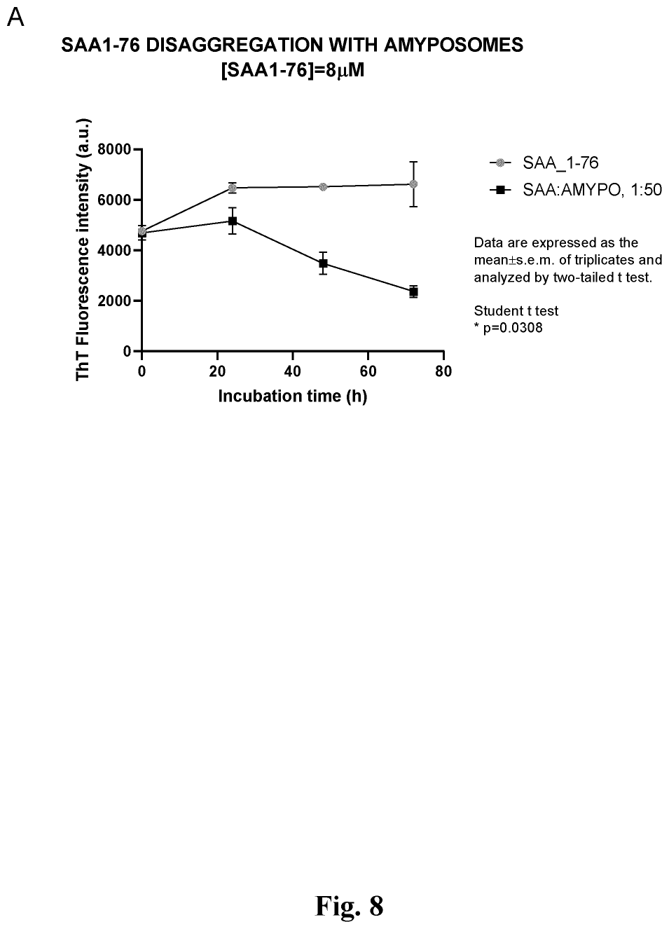

FIG. 8: Induction of SAA(1-76) fibrils disaggregation in vitro at 1:50 protein: Amyposomes ratio, investigated by ThioflavinT fluorescence spectroscopy. (A) time course of ThT fluorescence. a.u. =Fluorescence Intensity arbitrary units.

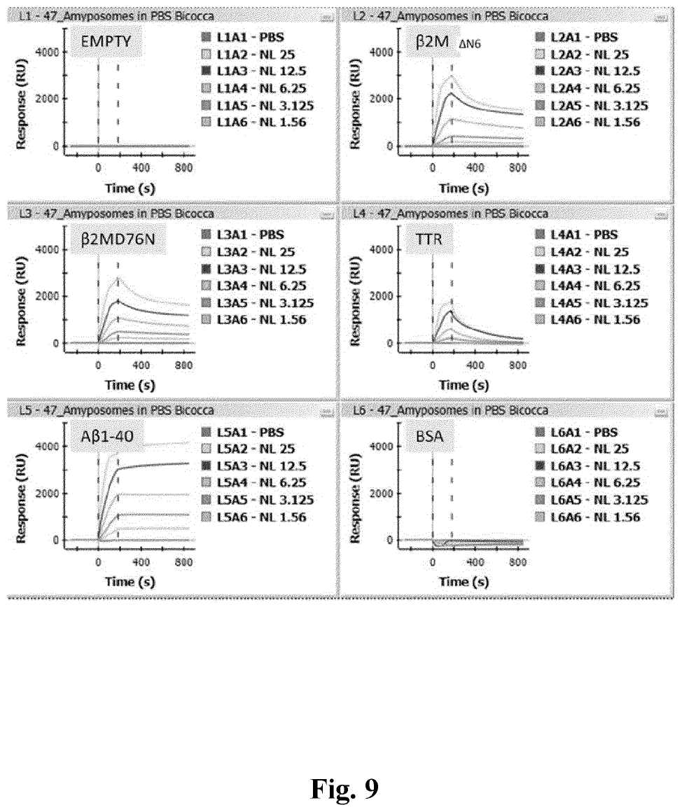

FIG. 9: SPR sensorgrams showing the specific binding of Amyposomes here indicated as nanoliposomes (NL) to the protein aggregates. Specific binding was obtained from the raw sensorgrams, by subtracting the non-specific binding measured in the empty surface, and the bulk effect observed with PBS alone. Note that after this double normalization, no specific binding could be detected on the surface coated with Bovine Serum Albumin (BSA), used here as a reference protein, whereas measurable and concentration-dependent binding signals were observed on the aggregates of the other proteins. NL were injected for three min, from t=0 to t=180s (as indicated by the vertical dotted lines). Running buffer (PBST) was then flowed from t=180s on, to evaluate the dissociation phase. The concentration of NL is indicated as μM of the exposed PA.

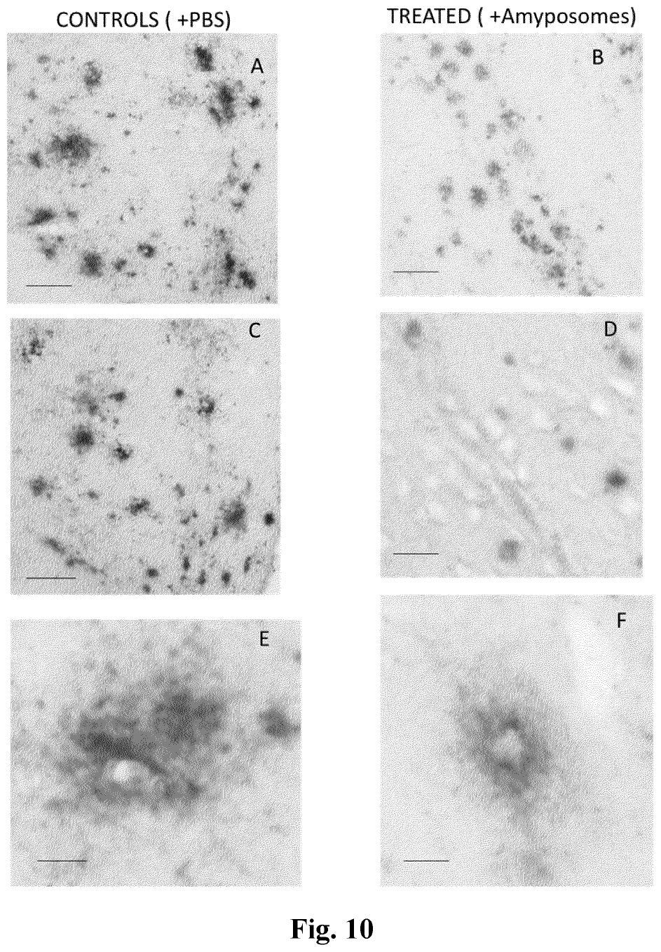

FIG. 10: Effect of Amyposomes treatment on vascular Aβ in the brain of Cerebral Amyloid Angiopathy Tg-SwDI mice. At the end of treatment, animals were sacrificed and brain was immunostained with the rabbit anti-Aβantibody for Aβvisualization. Representative brain sections of control mice treated with PBS (Panels A,C,E) and mice treated with Amyposomes. Scale bar =100 μm (A,B,C,D); 20 μm (E,F)

### DETAILED DESCRIPTION OF THE INVENTION

### Materials and Methods

The following reagents were purchased from Sigma- Aldrich: THIOFLAVIN T used as stain for amyloid (ThT , code T3516-25G), cholesterol Sigma grade (Chol, code C8667-5G), brain Sphingomyelin (SM, Code 860062P), Dimyristoylphosphatidic acid (PA, Code 830845P), Distearoyl-phospatidylethanolam ine-Polyetyleneglycl-maleim ide (DSPE-PEG-MAL, Code 880126P), 1,1,1,3,3,3-Hexafluoro-2-propanol (HFIP, Code 105228), Phenylmethylsulfonyl fluoride (PMSF, Code 10837091001). All other common reagents, resins for columns, solvent, reagents for electrophoresis and syntheses were also purchased from Sigma-Aldrich.

The Peptide CWGLRKLRKRLLR-NH2 (mApoE, Code 822594, SEQ ID No. 1) was purchased by KareBay Biochem — NJ USA. Trypsin Gold-Mass Spec Grade was purchased by Promega (code V5280).

### Proteins

Human Beta-Amyloid (1-40):
(Aβ-40, Code AS-24236, SEQ ID No. 2)
DAEFRHDSGYEVHHQKLVFFAEDVGSNKGAIIGLMVGGVV
was purchased from AnaSpect Inc.
Recombinant TTR variant TTR_S52P:
(SEQ ID No. 3)
MKHHHHH HPMSDYDIPT TENLYFE GAM GPTGTGESKC
PLMVKVLDAV RGSPAINVAV HVFRKAADDT WEPFASGKTS
EPGELHGLTT EEEFVEGIYK VEIDTKSYWK ALGISPFHEH
AEVVFTANDS GPRRYTIAAL LSPYSYSTTA VVTNPKE
was expressed and purified as described
previously [Verona G. et al. 2017 Sci. Rep. 7,
182-187].
Recombinant β2m variants:
β2m_D76N
(SEQ ID No. 4)
MIQRTPKIQVYSRHPAENGKSNFLNCYVSGFHPSDIEVDLLKNGERIEKV
EHSDLSFSKDWSFYLLYYTEFTPTEKNEYACRVNHVTLSQPKIVKWDRDM
and
β2m_ΔN6
(SEQ ID No. 5)
MIQVYSRHPAENGKSNFLNCYVSGFHPSDIEVDLLKNGERIEKVEHSDLS
FSKDWSFYLLYYTEFTPTEKDEYACRVNHVTLSQPKIVKWDRDM
were expressed and purified as described
previously [Valleix S. et al. Engl. J. Med., 2012,
366, 2276-2283; Verdone G. et al, Protein Sci.
2002, 11, 487-499; Esposito G. et al. Protein Sci.
2000, 9:831-45].
The truncated amyloidogenic form of Serum Amyloid
A, SAA(1-76):
(SEQ ID No. 6)
RSFFSFLGEA FDGARDMWRA YSDMREANYI GSDKYFHARG
NYDAAKRGPG GAWAAEAISDARENIQRFFG HGAEDS
was obtained by synthesis, as follows.

Briefly, three peptides:

Peptide 1
(SEQ ID No. 7)
H-RSFFSFLGEAFDG-NHNH₂
(segment 1-13)
Peptide 2
(SEQ ID No. 8)
H-CRDMWRAYSDMREANYIGSDKYFHARGNYDA-NHNH₂
(segment 14-14)
Peptide 3
(SEQ ID No. 9)
H-CKRGPG GAWAAEAISDARENIQRFFGHGAEDS-NH₂
(segment 45-76)

were synthesized through standard Fmoc-SPPS; each peptide was purified and subsequently the three fragments were assembled in sequence via hydrazide-based native chemical ligation method of peptide to obtain the complete protein SAA(1-76) [Zheng, J.S et al. Nature Protocol 2013, 8, 2483-2495]. Since no cysteine residue is present in the protein sequence, inventors replaced two alanine (at positions 14 and 45) with Cys and, after the complete assembly of the protein, the Cys residues were desulfurized to restore Ala [Wan, Q.; Danishefsky, S. J. Angew. Chem. Int. Ed. 2007, 46, 9248-9252].

The assembly of peptides was performed on Biotage Syro II peptide synthesizer [Sheppard, R. et al. J. Pept. Sci. 2003, 9, 545]. Fmoc (9-fluorenylmethyloxycarbonyI)- amino acids for synthesis of peptides were obtained from Iris-Biotech. All other protected amino acids and reagents for peptide synthesis were supplied by Sigma—Aldrich. The crude peptides were purified by RP-flash chromatography on Isolera Prime (Biotage) apparatus or preparative RP-HPLC. The purified fractions were characterized by HPLC-MS on Agilent 1200 equipped with Agilent 6130 MS.

Once the three peptides were purified through chromatographic techniques, the peptide hydrazide 1 was converted to thioester through NaNO₂ oxidation and thiolysis in presence of peptide 2 and 4-mercaptophenylacetic acid (MPPA). After the ligation was completed, the product was purified and the ligation reaction, involving fragment (1-44) and Peptide 3, was repeated to obtain the complete protein SAA (1-76). The product was purified by preparative HPLC on Vydac C18 column (10 ×250 mm, 10 μ, 300 Å), flow rate 3.7 mL/min, gradient elution 25-55% B over 30 min. The purity and correct composition of the product was confirmed by HPLC-MS analysis

### Preparation and characterization of Amyposomes®

Amyposomes® (Liposomes composed of Spingomyelin/cholesterol, functionalized with PA and with mApoE peptide (covalently linked to the outer surface) were synthesized as described [Balducci C. et al. J.Neurosci. 2014, 34: 14022-31, Bana et al., Nanomedecine 2013, doi:10.1016/j.nano.2013.12.001].

Briefly, Sphingomyelin and Cholesterol (1:1 molar ratio) were mixed with 2.5 molar % of mal-PEG-PE (also named, DSPE-mal, Sigma Aldrich 880126P-25MG) and with 5 molar% of phosphatidic acid (PA, Sigma Aldrich 830845P) in chloroform/methanol (2:1, v/v) and dried under a gentle stream of nitrogen followed by a vacuum pump for 3 h to remove traces of organic solvent. The resulting lipid film was rehydrated in phosphate-buffered saline containing 150 mM NaCI, pH 7.40 (PBS), vortexed and then extruded 10 times at 55° C. through a stack of two polycarbonate filters (100-nm pore size diameter) under 20 bar nitrogen pressure with an extruder. Liposomes were separated from possible unincorporated material by size-exclusion chromatography using PD-10 column and PBS as the eluent.

mApoE peptide was added to Liposomes in PBS to give a final peptide:mal-PEG-PE (or DSPE-PEG-MAL) molar ratio of 1.2:1 and incubated overnight at room temperature to form a thioether bond with mal-PEG-PE (or DSPE-PEG-MAL) since mApoE peptide reacts only with the portion of mal-PEG-PE present in the outer leaflet of the liposomes (50-60% of the total).

Liposomes composed of sphingomyelin/cholesterol, functionalized with PA and covalently linked with the peptide mApoE (Amyposomes®) were separated from unbound peptide using PD-10 column. The yield of coupling of the peptide to liposomes was assessed by measuring the tryptophan fluorescence intensity (λex =280 nm) of the incubation mixture and of Amyposomes® recovered from the PD-10 column. Spectra were recorded between 300 and 450 nm using a Cary Eclipse spectrofluorometer (Varian). The amount of peptide bound to liposomes was calculated from the Tryptophan (present in the peptide) fluorescence intensity of a known amount of the peptide dissolved in PBS, taken as the standard. Lipid recovery was measured by Stewart's assay [Stewart J.C., Anal. Biochem. 1980, 104, 10-14].

Preferred liposomes have the following composition

46,25 mol % cholesterol
46,25 mol % sphingomyelin
1.25-1,5% mol mal-PEG-PE linked to mApoE
1.25-1,0% mol mal-PEG-PE free
5 mol % phosphatidic acid

Wherein the sum of the % of mal-PEG-PE free and % mal-PEG-PE linked to mAPOE is 2,5%.

More preferred liposomes have the following composition: 46,25 mol% cholesterol 46,25 mol% sphingomyelin 2,5% mol mal-PEG-PE 5 mol% phosphatidic acid

These liposomes are functionalized on surface with 1,25 mol% of mAPOE. Liposomes size, Polidispersity Index (PDI) and ζ-potential were characterized as described previously [Gobbi M, et al. Biomaterials 2010;31:6519-29]. Liposomes without PA and mApoE (Plain) were used as controls.

### Aggregation/Disaggregation Assay

Inhibition of protein aggregation or destabilization of preformed aggregates by Amyposomes was monitored essentially as described [ Bana L. et al., Nanomedicine. 2013 ,10:1583-90] by using the Thioflavin T (ThT) fluorescence assay, which identifies amyloid containing β-sheet structures. Fluorescence (λecc =450 nm; λem =480 nm) was measured with Wallac 1420 Victor2 spectrofluorometer (Perkin Elmer). Data were subtracted from fluorescence of Amyposomes alone.

ThT is a weakly fluorescent probe in water, but its fluorescence increases when it intercalates among the stacked β-sheets of aggregated amyloid proteins molecules. Therefore, the increase of ThT fluorescence during time in the presence of a protein, can be taken as a parameter related to the increased extent of protein aggregation.

On the other side, when ThT molecules entrapped within aggregates are released in water, as a result of disaggregation, then the fluorescence decreases. Therefore, the decrease of fluorescence over time can be taken as a parameter related to the decreased aggregation (or “increased disaggregation”).

To monitor the possible inhibiting effect of Amyposomes on protein aggregation, the non-aggregated form of proteins was added with 10 μM ThT and with different amounts of Amyposomes® directly in Costar 96-well black plates. The change in fluorescence was monitored continuously during time with Wallac 1420 Victor2 spectrofluorometer (Perkin Elmer). Alternatively, instead of continuously following the fluorescence, the non-aggregated form of proteins was added with different amounts of Amyposomes® and, at different times of incubation, an aliquot of the samples was withdrawn, added with 10 μM ThT, and the fluorescence measured as above described.

To monitor the possible destabilizing effect of Amyposomes on protein aggregates, the pre-aggregated form of proteins was added with 10 μM ThT and with different amounts of Amyposomes®, then the time course of fluorescent was followed as above described.

### Atomic Force Microscopy

Aliquots of 10 μI amyloid fibrils obtained as described in Materials and Methods were allowed to adhere onto freshly cleaved mica for 10 min. The samples were washed 3 times with 200 μL Milli-Q water and air dried overnight. AFM was performed Tapping Mode in air using stiff silicon cantilevers (RTESP-Veeco, resonant frequencies ˜300 kHz, spring constant ˜40 N/m). AFM images were acquired at a scan rate of 1 Hz with a Nanowizard II (JPK Instruments, Berlin, Germany). Images were obtained by scanning the samples at a rate of 0.5-1.0 Hz, and 512 x 512 pixels were collected in each image and analysed using JPKs software. Samples were exhaustively examined to confirm their homogeneity.

### Aggregation-Disaggregation Protocol for Aβ40

### Preparation of Disaggregated Aβ40.

Lyophilized peptide was stored in sealed glass vials at −80 ° C. Prior to resuspension, each vial was allowed to equilibrate to room temperature for 30 min to avoid condensation upon opening the vial. Each vial of peptide was diluted in 100% HFIP to 1 mM using a glass gas-tight Hamilton syringe with a Teflon plunger and incubated under stirring for 30 min at RT. The HFIP was allowed to evaporate in the fume hood, and the resulting clear peptide films were dried under vacuum (6.7 mtorr) in a SpeedVac (Savant Instruments) and stored desiccated at −20° C. DMSO was added to solubilize the peptide film in order to obtain a solution disaggregated Aβ40 at 5 mM protein concentration. Then, the effect of Amyposomes® on protein aggregation was investigated by the ThT assay.

### Preparation of Aggregated Aβ40.

Fibrils were prepared by diluting 5 mM Aβ40 in DMSO to 25 μM in PBS, immediately vortexing for 30 s, and incubating at 37° C. or 7 days.

Then the effect of Amyposomes® on protein fibrillary aggregates was investigated by the ThT assay (as described in methods above).

### Aggregation-Disaggregation Protocol for TTR S52P

Recombinant S52P_TTR, at 35 μM concentration in 200 μl PBS (150 mM NaCI, pH 7.4) was incubated at 37° C. in Costar 96-well black plates in the presence of trypsin, at an enzyme:substrate ratio of 1:50, w/w to hydrolytically generate the amyloidogenic form of the protein. To monitor the influence of Amyposomes on protein aggregation, 1.5 mM of PMSF was added after 6h incubation, to inhibit the trypsin enzymatic activity. Under these conditions, the amyloidogenic form of the protein is generated but its aggregation into fibrils has not yet occurred. Afterwards, Amyposomes were added and the fibrillization process was followed by the ThT fluorescence assay, carried out as described above.

To monitor the effect of Amyposomes on disaggregation, S52P_TTR was incubated in the presence of trypsin, at an enzyme:substrate ratio of 1:50, w/w, to hydrolytically generate the amyloidogenic form of the protein. After 24h incubation, when formation of fibrils has already occurred, 1.5 mM of PMSF was added to inhibit the trypsin enzymatic activity. Afterwards, Amyposomes were added, and the disaggregation process was followed by ThT fluorescence assay, carried out as above described.

### Aggregation-Disaggregation Protocol for β2 -Microglobulin (D76 N and ΔN6 variants)

The inhibition of β2-microglobulin aggregation by Amyposomes was investigated by the ThT assay, carrying out incubation of 40 μM recombinant β2-Microglobulin in 200 μl of PBS at 37° C. in the presence of different amounts of Amyposomes.

To monitor the effect of Amyposomes on protein disaggregation, 40 pM of recombinant β2-microglobulin in PBS was previously incubated at 37° C., under constant stirring to allow the fibril formation. After 24h incubation, ThT disaggregation assays were carried out in the presence of different amounts of Amyposomes.

### Statistical analysis

Data of ThT assays were normalized to the ThT signal of reference samples containing all the reagents but lacking the amyloid protein.

### EXAMPLES

### Example 1: Characterization of Amyposomes®

The size of Amyposomes used within these experiments was 157 ±22.1 nm with a Polydispersity Index (PDI) of 0.072 (average of 15 different batches measured in triplicate).

### Example 2: Aggregation-Disaggregation of A β40

As inferred from the values of the ThT fluorescence, immediately after that the disaggregated form Abeta40 was incubated in PBS, aggregation started and reached a maximum after 48 hours, then it remained constant.

In the presence of Amyposomes at different protein:Amyposomes ratio, a biphasic behaviour was observed: after an initial phase (24 h) during which an increase of aggregation was observed, then the aggregation decreased constantly over time and, in the case of 1:50 ratio, almost only disaggregated Abeta 40 was present after 4 days. The results are reported in FIG. 1A and 1B.

The influence of Amyposomes on disaggregation of Aβ40 was evaluated on preparations of previously aggregated Aβ40. The results are reported in FIG. 2A and 2 B. The aggregated form of the peptide was stable for at least 7 days in the absence of Amyposomes. In the presence of Amyposomes at different peptide:Amyposomes ratios, with the exception of plain liposomes, the fibrils disaggregated, following a seemingly hyperbolic course. The disaggregation increased on increasing the concentration of Amyposomes. In the case of peptide:Amyposomes 1:50 ratio, only 10% residual of aggregates were present after 5 days. However, already after 24 h, 40% residual of aggregates was apparent.

AFM imaging (FIG. 2C), showed fibril chains unbranched, slightly curved, and elongated. Such elongated fibrils exhibit an apparent height of 6 nm. After 3h of incubation with Amyposomes, AFM observations show disordered small aggregates constituted by fibrils fragments, typical of fibrils dissolution (FIG. 2D).

These results support the therapeutic effect of the liposomes of the present invention for the treatment and/or prevention of CAA.

### Example 3: Aggregation-Disaggregation of TTR_S52P

Aggregation of TTR was triggered by addition of trypsin, catalyzing cleavage of a peptide fragment from the native protein [Mangione P.P. et al. J. Biol Chem. 2018, 293, 14192. Addition of a trypsin inhibitor after 5h (performed to prevent the possible hydrolysis of mApoE upon successive addition of Amyposomes) did not affect the aggregation, that started after 5h and reached a maximum 7 h later then remaining constant.

To study their influence on aggregation, Amyposomes were added to the protein immediately after the trypsin inhibitor and, in their presence, a reduction of the aggregation was observed. The reduction was higher at higher Amyposome amounts and, at a TTR: Amyposomes ratio of 1:50, the aggregation was 40% with respect to the protein in the absence of Amyposomes. The results are reported in FIG. 3A, B.

The influence of Amyposomes on disaggregation of TTR was evaluated on preparations of previously aggregated protein. The protein in the aggregated form was stable at least for 5 h.

In the presence of Amyposomes, the fibrils disaggregated. The higher the amount of Amyposomes, the higher was the disaggregation of the protein, so that in the case of TTR:Amyposomes 1:50 ratio, only a 5% residual of aggregates was observed after 5 h. The results are reported in FIG. 4.

Imaged by AFM, TTR S52P produced morphologically typical mature amyloid fibrils, 4-7 nm in height emerging from a thick layer of short fibrils, geometrically ordered (FIG. 4C). After 3h of incubation with Amyposomes there was a strong reduction of fibrils with a change of fibrils order and geometry (FIG. 4D). This demonstrates a dissolution of fibrils mediated by Amyposomes.

These results support the therapeutic effect of the liposomes of the present invention for the treatment and/or prevention of SSA and FAP.

### Example 4: Aggregation-Disaggregation of β2 Microglobulin

Two variants of the protein (D76N and ΔN6) were investigated.

The amyloidogenic variant of β2-microglobulin, D76N, is associated with a familial form of the disease and is characterized by progressive bowel disfunction and extensive amyloid deposits in the spleen, liver, heart, salivary glands and nerves. ΔN6 is a ubiquitous constituent of β2-m amyloid deposits in patients affected by dialysis-related amyloidosis and, due to its capacity to act as a seed in the fibrillogenesis of full length β2-m, it could have a crucial role in dictating the clinical history of the disease.

The aggregation of D76N followed a biphasic behaviour: after 10 h incubation the aggregation started and increased slowly up to 19 h, then abruptly increased reaching a maximum at 21 h, then remained constant. On the other side, kinetics for ΔN6 variant were much faster, the aggregation starting immediately upon incubation, and reaching a maximum in 2 h, then remaining constant (FIG. 5).

To study their influence on aggregation, Amyposomes were incubated with the protein, and a reduction of the aggregation was observed. The reduction increased on increasing the Amyposome amount and, at a D76N:Amyposomes ratio of 1:50, the final aggregation extent was 36% with respect to the protein alone. In the case of ΔN6, at a protein:Amyposomes ratio of 1:50, the aggregation extent was 20% with respect to the protein alone. The results are reported in FIGS. 5 and 6.

The influence of Amyposomes on disaggregation of β2 Microglobulin was evaluated on preparations of previously aggregated protein.

In the case of D76N, the protein in the aggregated form was stable at least for 3 days. ln the presence of Amyposomes, a decrease of fluorescence was observed starting at 24 h of incubation that decreased constantly up to 72 h. This effect was not evident at 1:2 protein: Amyposomes ratio, was minimal at 1:10 ratio, and was stronger, and comparable, at 1:30 and 1:50 ratios, leading to only a 15% residual aggregates after 3 days.

In the case of ΔN6, a decrease of fluorescence was observed starting immediately after incubation in the presence of Amyposomes, and the kinetics was much faster, the fluorescence reaching a minimum after 2 h. The effect increased on increasing the amount of Amyposomes. At 1:50 ratios, 50% residual aggregates were present. The results are reported in FIGS. 7 and 8.

These results support the therapeutic effect of the liposomes of the present invention for the treatment and/or prevention of all diseases associated with β2-microglobulin accumulation or deposit, comprising all dialysis-related amyloidosis, including familial form of dialysis-related amyloidosis, characterized by progressive bowel disfunction and extensive amyloid deposits in the spleen, liver, heart, salivary glands and nerves and sporadic forms of dialysis-related amyloidosis.

### Example 5: Aggregation-Disaggregation of SAA(1-76)

SAA aggregation started after 48 hours incubation. Amyposomes were added to the protein and, in their presence, a strong reduction of the final aggregation extent was observed. The reduction increased on increasing the Amyposome amount and, at a TTR: Amyposomes ratio of 1:50, only disaggregated for was present. The results are reported in FIG. 9.

The influence of Amyposomes on disaggregation of SAA(1-76) was evaluated on preparations of previously aggregated protein.

In the presence of Amyposomes, at 1:50 ratio the extent of aggregation decreased and after 72 h incubation 30% residual of aggregates was observed. The results are reported in FIG. 10.

These results support the therapeutic effect of the liposomes of the present invention for the treatment and/or prevention of reactive amyloidosis, even associated to rheumatoid arthritis, atherosclerosis or for the treatment and/or prevention of end stage renal failure in patients with type 1 and 2 diabetes mellitus and diabetic kidney disease.

### Example 6: Evaluation of the Binding of Amyposomes to Aggregates of Four Different Amyloidogenic proteins by Surface Plasmon Resonance (SPR)

The binding properties of the Amyposomes for the aggregates of four different amyloidogenic proteins: amyloid-β1-40 (Aβ1-40), transthyretin (TTR), and β2- microglobuline (β2M), in two mutated forms: β2MD76N and β2MΔN6 by SPR were evaluated.

### METHOD

Amyposomes were flowed, at different concentrations (1.56, 3.125, 6.25, 12.5, 25 pM) of PA exposed on Amyposomes surface, over a chip surface coated with the protein aggregates (30 μg/mL, in acetate buffer, pH 4), following the same design previously used to demonstrate the binding of flowing liposomes to immobilized Aβ1-42 fibrils (Gobbi et al., 2010, Biomaterials 31: 6519). Bovine Serum Albumin was used as a negative control. The sensorgrams (time course of the SPR signal in Resonance Units, RU, FIG. 9) were normalized to a baseline value of 0. The signals observed in the surfaces immobilizing protein aggregates was corrected by subtracting the nonspecific response observed in the reference surfaces, as indicated. When appropriate, the sensorgrams were fitted using the ProteOn analysis software to obtain the association and dissociation rate constants (kon and koff) and the equilibrium dissociation constant (KD).

### RESULTS

Amyposomes have no “specific” binding signal on BSA (i.e. the reference protein), even at the highest concentration tested. The estimated (see above) equilibrium dissociation constants (KD) were:

0.12 μM for Aβ1-40 fibrils;
1.75 μM on average for the two β2M aggregates;
14.2 μM for TTR aggregates.

These differences in the KD values were mainly due to differences in the dissociation rate constants, whereas the association rate contants were not markedly different.

The present SPR data (FIG. 9) show that Amyposomes bind, in a concentration-dependent manner, the aggregates of Aβ1-40, β2MΔN6, β2MD76N and TTR. This binding is specific since it was not observed with immobilized BSA.

### Example 7: Effect of Treatment with Amyposomes on Vascular aβ in Cerebral Amyloid Angiopathy (CAA) Animal Models

### MATERIALS

Common Reagents and reagents for immunohistochemistry were from Merck. Primary rabbit AβAntibodies (Catalog # 71-5800, INVITROGEN) and secondary anti-rabbit antibodies (BA-1000, VINCI-BIOCHEM) were from Thermo Fisher Scientific. Cerebral Amyloid angiopaty Mouse model was from Jackson Laboratories (Cat. # Stock :7027C57BL/6-Tg(Thyl-APPSwDutlowa)BWevn/Mmjax).

Mouse model utilized to study Cerebral Amyloid Angiopathy :Tg-SwDI mice.

The model used to evaluate the effect of Amyposomes is the triple transgenic C57 / 6-Tg (Thyl APPSwDutlowa) BWevn / Mmjax Hem izygous1,2 (Tg-SwDI mice). This mouse model has been primarily designed to study CAA (Jakel L. Animal Models of Cerebral Amyloid Angiopathy Clin. Sci. 2017 131:2469).

These mice express human APP770 containing the Swedish (K670N / M671L) ,Dutch (APP E693Q), Iowa (APP D694N) mutations under control of the mouse Thyl promoter. In this model, the fibrillary microvascular accumulations of Aβ begin at about 3 months of age. At 12 months 50% of the brain microvasculature has Aβ deposits increasing to 85-90% at the age of 24 months. Higher levels of Aβ₄₀ compared with Aβ₄₂ have been measured in isolated cerebral microvessels. Accumulation of Aβ in parenchima is diffuse. (Miao J. Am. J. Pathol. 2005).

These mice features mirror human Cerebral Amyloid Angiopathy. In fact, human brains affected by CAA show few parenchymal amyloid plaques while vascular Aβdeposits comprise predominantly Aβ40 (Suzuki,N et al. 1994 High tissue content of soluble Abeta1-40 is linked to cerebral amyloid angiopathy Am. J. Pathol . 145:452; Herzig MC, Nat Neurosci. 2004;7:954-60). Of note, parenchymal senile plaques in AD are composed principally of Aβ1- 42 (Dickson,D.W., et al. 1988 Alzheimer's disease. A double-labeling immunohistochemical study of senile plaques. Am. J. Pathol. 132, 86),

Evaluation of the effect of Amyposomes treatment on vascular Abeta in CAA mouse model Tg-SwDl.

SwDI mice (n=10, aged 6 months) were treated with 3 intraperitoneal injections /week (1 injection every other day) for 3 weeks. Each injection contained 2.6 mg of Amyposomes / 100μL of PBS. SwDI animals (n=10, aged 6 months) treated with PBS (100μL/injection) following the same scheme of treatment were used as control. The animals were sacrified and brain was post-fixed in paraformaldehyde (4%) for histology studies.

Vascular Aβ deposition was examined using rabbit Anti-Abeta antibodies. To this purpose brain cryostat sections (30 μm) were incubated for 1 h at room temperature with the primary antibody (rabbit Anti-Aβ, 1:200, Catalog # 71-5800, INVITROGEN). After incubation with the anti-rabbit biotinylated secondary antibody (1:200; 1 h at room temperature, Catalog # BA-1000, VINCI-BIOCHEM), immunostaining was developed using the avidin— biotin kit (PK4000, Vector Laboratories) and diaminobenzidine (D8001, Sigma, Italy).

As shown in FIG. 10, the vascular deposition of Aβ was evident in all analyzed control mice (treated with PBS). A reduction of vascular deposition of Aβ was clearly evident in all the mice treated with Amyposomes. Representative images of control vs.

Amyposome-treated animals are reported in Fig . 10.

1. A method for the treatment and/or prevention of amyloidosis, wherein said amyloidosis is not Alzheimer' s disease and is selected from the group consisting of: senile systemic amyloidosis, familial amyloid polyneuropathy, dialysis-related amyloidosis, reactive amyloidosis, cerebral amyloid angiopathy, prion diseases, Finnish type amyloidosis, leptomeningeal amyloidosis, Familial visceral amyloidosis, Primary cutaneous amyloidosis, Prolactinoma, Familial corneal amyloidosis, Senile amyloid of atria of heart, Medullary carcinoma of the thyroid LECT2 amyloidosis, type 2 diabetes mellitus, end stage renal failure in patients with type 1 and 2 diabetes mellitus and diabetic kidney disease, comprising administering an effective amount of a liposome comprising: a lipid or lipid mixture; phosphatidic acid and/or cardiolipin and apolipoprotein E or a fragment thereof to a patient in need thereof and wherein said lipid or lipid mixture is selected from the group consisting of: sphingomyelin, phosphatidylcholine, phosphatidylethanolamine and cholesterol.

2. The method according to claim 1 wherein the lipid is a mixture of sphingomyelin and cholesterol.

3. The method according to claim 1, wherein the apolipoprotein E is any of the two isoforms E2, E3 of ApolipoproteinE or a fragment thereof.

4. The method according to claim 1, wherein said apolipoprotein E includes, at its C-terminal, a cystein-ending tripeptide.

5. The method according to claim 1, wherein the liposome further comprises at least one PEG (polyethyleneglycol) molecule, PEO (poly-ethylene-oxide) molecule, POE (poly-oxy-ethylene) molecule, PDO (Polydioxanone) molecule or a mixture thereof, the average molecular mass of the PEG molecule optionally being above 1 kDa but less than 1 lkDa.

6. The method according to claim 5 wherein the PEG molecule is selected from the group consisting of : methylpolyethyleneglycol-1,2-distearoyl-phosphatidyl ethanolamine conjugate (MPEG-2000-DSPE); monomethoxypolyethylene glycol (MPEG-OH), monomethoxypolyethylene glycol-succinate (MPEG-S), monomethoxypolyethylene glycol-succinimidyl succinate (MPEG-S -NHS), monomethoxypolyethylene glycol-amine (MPEG-NH2), monomethoxypolyethylene glycol-tresylate (MPEG-TRES), and monomethoxypolyethylene glycol-imidazolyl-carbonyl (MPEG-IM); or mixtures thereof.

7. The method according to claim 1, wherein the phosphatidic acid is present in 1-20% molar percentage.

8. The method according to claim 1, wherein the apolipoprotein E is present in 1-5% molar percentage.

9. The method according to claim 1, wherein said liposome consists of:
46.25 mol % cholesterol
46.25 mol % sphingomyelin
1.25-1.5% mol mal-PEG-PE linked to mApoE
1.25-1.0% mol mal-PEG-PE free; and
5 mol% phosphatidic acid
wherein the sum of the % of mal-PEG-PE free and % mal-PEG-PE linked to mAPOE is 2.5%
wherein mal-PEG-PE is 1,2 stearoyl-sn-glycero-3- phosphoethanolamine-N- [maleimide(poly(ethylene glycol)-2000)] and mAPOE is SEQ ID. No. 1.

10. The method according to claim 1, wherein the liposome has an average size <200 nm.

11. The method according to claim 1, wherein the liposome has a PDI <0.2.

12. The method according to claim 1, wherein the liposome decreases amyloid protein aggregation and/or increases amyloid protein disaggregation.

13. The method according to claim 12 wherein the amyloid protein is selected from the group consisting of: Transthyretin , β₂microglobulin, amylin, amyloid light chain, Serum amyloid A protein, Gelsolin, Cystatin C, ApoA1, Fibrinogen alfa chain, LYZ (Lysozyme, also known as muramidase or N-acetylmuramide glycanhydrolase), OSMR (Oncostatin-M specific receptor subunit beta also known as the Oncostatin M receptor), Integral membrane protein 2B (ITM2B or BRI2), prolactin, LECT2 protein, keratoepithelin (Transforming growth factor, beta-induced, 68kDa, also known as TGFBI (initially called BIGH3, BIG-H3), calcitonin, atrial natriuretic factor and prion protein.

14. A method for the treatment and/or prevention of amyloidosis, wherein said amyloidosis is not Alzheimer's disease and is selected from the group consisting of: senile systemic amyloidosis, familial amyloid polyneuropathy, dialysis-related amyloidosis, reactive amyloidosis, cerebral amyloid angiopathy, prion diseases such as Creutzfeldt-Jakob disease (humans), BSE or “mad cow disease” (cattle), and scrapie, Finnish type amyloidosis, leptomeningeal amyloidosis, Familial visceral amyloidosis, Primary cutaneous amyloidosis, Prolactinoma, Familial corneal amyloidosis, Senile amyloid of atria of heart, Medullary carcinoma of the thyroid LECT2 amyloidosis, type 2 diabetes mellitus, end stage renal failure in patients with type 1 and 2 diabetes mellitus and diabetic kidney disease, comprising administering a pharmaceutical composition comprising a liposome comprising a lipid or lipid mixture; phosphatidic acid and/or cardiolipin and apolipoprotein E or a fragment thereof, together with one or more pharmaceutically acceptable excipients and, optionally, further active agents to a patient in need thereof.

15. The method of claim 1, wherein the reactive amyloidosis is accompanied by rheumatoid arthritis and/or atherosclerosis.

16. The method of claim 3, wherein the fragment is one of:
a) the amino acid sequence 100-200 of ApoE;
b) within the amino acid sequence 120-170 of ApoE; or
c) the sequence 141-150 of ApoE or a dimer thereof.

17. The method of claim 4, wherein the cysteine-ending tripeptide is CWG.

18. The method of claim 4, wherein the apolipoprotein E has the sequence CWGLRKLRKRLLR or is a dimer thereof.

19. The method of claim 7, wherein molar percentage for the phosphatidic acid is 1-10%.

20. The method of claim 8, wherein the molar percentage for the apolipoprotein is 1-3%.


SEQUENCE LISTING (12 sequences)

SEQ ID NO: 1 - protein, 13 aa, Artificial Sequence
  synthetic
CWGLRKLRKRLLR

SEQ ID NO: 2 - protein, 40 aa, Artificial Sequence
  synthetic
DAEFRHDSGYEVHHQKLVFFAEDVGSNKGAIIGLMVGGVV

SEQ ID NO: 3 - protein, 154 aa, Artificial Sequence
  synthetic
MKHHHHHHPMSDYDIPTTENLYFEGAMGPTGTGESKCPLMVKVLDAVRGSPAINVAVHVFRKAADDTWEPFASGKTSEPGELHGLTTEEEFVEGIYKVEIDTKSYWKALGISPFHEHAEVVFTANDSGPRRYTIAALLSPYSYSTTAVVTNPKE

SEQ ID NO: 4 - protein, 100 aa, Artificial Sequence
  synthetic
MIQRTPKIQVYSRHPAENGKSNFLNCYVSGFHPSDIEVDLLKNGERIEKVEHSDLSFSKDWSFYLLYYTEFTPTEKNEYACRVNHVTLSQPKIVKWDRDM

SEQ ID NO: 5 - protein, 94 aa, Artificial Sequence
  synthetic
MIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 6 - protein, 76 aa, Artificial Sequence
  synthetic
RSFFSFLGEAFDGARDMWRAYSDMREANYIGSDKYFHARGNYDAAKRGPGGAWAAEAISDARENIQRFFGHGAEDS

SEQ ID NO: 7 - protein, 13 aa, Artificial Sequence
  synthetic
RSFFSFLGEAFDG

SEQ ID NO: 8 - protein, 31 aa, Artificial Sequence
  synthetic
CRDMWRAYSDMREANYIGSDKYFHARGNYDA

SEQ ID NO: 9 - protein, 32 aa, Artificial Sequence
  synthetic
CKRGPGGAWAAEAISDARENIQRFFGHGAEDS

SEQ ID NO: 10 - protein, 10 aa, Artificial Sequence
  synthetic
LRKLRKRLLR

SEQ ID NO: 11 - protein, 20 aa, Artificial Sequence
  synthetic
LRKLRKRLLRLRKLRKRLLR

SEQ ID NO: 12 - protein, 23 aa, Artificial Sequence
  synthetic
CWGLRKLRKRLLRLRKLRKRLLR